(19) United States
(12) Patent Application Publication
(10) Pub. No.: US 2017/0146512 A1
(43) Pub. Date: May 25, 2017

(54) METHOD OF DETERMINING CONCENTRATION VALUES OF AN ANALYTE

(71) Applicant: B. BRAUN AVITUM AG, Melsungen (DE)

(72) Inventors: MIRAN ALIC, Melsungen (DE); Christof Strohhöfer, Kassel (DE); Waldemar Janik, Melsungen (DE)

(21) Appl. No.: 15/359,253

(22) Filed: Nov. 22, 2016

(30) Foreign Application Priority Data
Nov. 23, 2015 (DE) 10 2015 120 216.6

(51) Int. Cl. G01N 33/49

(52) U.S. Cl. CPC G01N 33/49; G01N 21/33

(57) ABSTRACT

A method for calibrating a measurement signal and/or for tracking a quantitative variable comprises the measuring of an analyte which exists in a solution with a certain concentration and has predetermined decay kinetics and the generating of a continuous measurement signal having decay kinetics at least corresponding to those of the analyte. The decay kinetics of the measurement signal and the decay kinetics of the analyte are correlated using at least one predetermined calibration point of both decay curves. Subsequent concentration values of the analyte are then calculated from the measurement signal.

### CROSS REFERENCE TO RELATED APPLICATIONS

This application claims priority to German application DE 10 2015 120 216.6 filed Nov. 23, 2015, the contents of such application being incorporated by reference herein.

### FIELD OF THE INVENTION

The invention relates to a method for calibrating a measurement signal and for tracking a quantitative variable.

### BACKGROUND OF THE INVENTION

In the quantitative measurement of chemical components, for instance analytes such as the uremic toxins creatinine and urea, additives are frequently used which serve for ensuring the uniqueness of substances such as for instance enzymes, colorants or nanoparticles, or of substances which alter the general ambient characteristics of the measuring solution, for instance its pH value. Such additives may be specialized in analytes (i.e. “target” these). The use of additives has the consequence that a certain amount of additives has to be stocked in a corresponding device. This is involved with costs.

The calculation of concentrations of selected substances is usually carried out by the exploitation of a measurand, for instance a conductivity (for electrolytes, for example) or an extinction (for creatinine and/or urea, for instance). Here, the measurement signal is frequently a superposition of contributions of various substances. In this case, the task of the measuring system is to extract the variable to be measured from a corresponding measurement signal and hence to isolate it from the interfering substances. In practice, this is performed for instance by the use of a combination of chemical additives (enzymes) and colorants. The alteration of the interaction between the enzyme and the colorant is then proportional to the concentration of the searched substance, allowing a simple conversion. It is disadvantageous, however, that each measurement of a concentration requires the use of enzymes and colorants.

In particular in a dialysis in which the dialyzer brings about a substance transfer between the blood and the dialysis liquid and which can be described in simplified form as a process in which substances (uremic toxins, electrolytes) from the blood are transported via a membrane (dialyzer) into a preprocessed liquid (dialysis liquid), a continuous use of additives is problematic. The used dialysis liquid (dialysate) as a buffer solution obtained in the dialysis, in which many measurements of measurement signals are carried out, is a chemical image of the blood and loaded with a high number of uremic toxins. Usually, optical sensors are used here in order to calculate a removal rate (e.g. Kt/V). If a corresponding additive is available, said sensors also allow to obtain qualitative magnitudes such as for instance the concentration of an analyte. However, the use of additives is very cumbersome in this environment and their storage is so effortful that a quantitatively measuring sensor could not become established in dialysis heretofore.

### SUMMARY OF THE INVENTION

Thus, the invention relates to the object to eliminate the aforementioned disadvantages and to provide a method with which a saving of additives can be achieved with a (quasi) continuous quantitative measurement of substances.

This object is solved according to aspects of the invention by a method having the features of the independent claim. Advantageous developments/embodiments of the invention are subject-matter of the attached sub-claims.

The invention relates to the general idea to allow the calculation of subsequent concentration values by tracking a raw signal through a suitable mapping of a concentration value on a signal value at the outset of a therapy (calibration). Apart from the addition of an additive at the beginning of a therapy (i.e. at least a single addition), no further detection of a signal is required in a surrounding which is altered (for instance with respect to its pH value). However, it is readily possible to take further measurements. In this case, it is possible to determine further variables such as for instance the distribution volume or transfer impairment mechanisms.

In line with the aforementioned basic idea, a single measurement of a concentration at the outset of a therapy may be sufficient to determine subsequent concentration values without the use of additives with a suitable calibration while utilizing the properties to the effect that the removal of small-molecular substances from e.g. blood in for instance a used dialysis liquid follows a defined kinetic scheme. Thus, the aforementioned disadvantages can be avoided by a skillful calibration.

The invention is advantageous in particular to the effect that a reduced use of additives is required, cost and material savings are realized, the wear and tear will be lower and a higher security is obtained.

Specifically, the above advantages are realized and the object is solved by a method for calibrating a measurement signal and/or for tracking of a quantitative variable, comprising: measuring an analyte which exists in a solution with a certain concentration and has predetermined decay kinetics and generating a continuous measurement signal having decay kinetics at least corresponding to those of the analyte; correlating the decay kinetics of the measurement signal and the decay kinetics of the analyte using at least one predetermined calibration point of both decay curves; and calculating subsequent concentration values of the analyte from the measurement signal.

In other words the above advantages are realized and the object is solved by a method for calibrating a measurement signal and/or for tracking a quantitative variable, comprising: generating at least one measured value of an analyte, which exists in a solution and has a predetermined decay curve defining a decaying concentration course of the analyte, as a quantitative variable; generating an at least quasi-continuous measurement signal having a decaying course at least corresponding to the decaying concentration course of the analyte based on a sensor signal, wherein the analyte determines/defines a portion of the measurement signal and a remaining portion of the measurement signal is not determined by the analyte; correlating the course of the measurement signal and the decay curve of the analyte using at least one predetermined calibration point on the decay curve of the analyte and the course of the measurement signal respectively; and tracking the quantitative variable by calculating subsequent concentration values of the analyte from the measurement signal.

Preferably, the further following method steps are carried out: selecting a first measurement signal course obtained for a first quantitative variable among a plurality of measurement signal courses obtained for the first quantitative variable and for at least one second quantitative variable; carrying out a calibration for at least one measured value of a second measurement signal course with the second quantitative variable from the measurement of the corresponding measured value with the first quantitative variable; checking all measured values obtained with the first quantitative variable and the second quantitative variable in terms of a linear or at least sufficiently linear connectedness; and whenever there is a linear or at least sufficiently linear connectedness, calculating at least one subsequent measured value for the second quantitative variable from the first measurement signal course for the first quantitative variable.

Preferably, the first quantitative variable is a first pH value, the second quantitative variable is a second pH value, and the measured values are extinction values.

Preferably, the first and the second measurement signal course is based on the same wavelength of an analyzing light used for the determination of the extinction values.

Preferably, for calculating a distribution volume the following method steps are further carried out: measuring the solution-side concentration of a desired analyte present in the solution; correlating an extinction signal and/or at least one extinction value with the measured concentration; measuring the extinction signal and/or at least one extinction value as a function of a time elapsing during a therapy and obtaining a continuously available measurement signal; calculating a concentration signal and/or at least one concentration value at predetermined discrete points in time from the continuously available measurement signal; measuring the blood-side concentration of the analyte; determining a removed overall mass; and determining the distribution volume from the determined overall mass, the solution-side concentration of the analyte and the blood-side concentration of the analyte.

Preferably, the distribution volume is calculated in accordance with the relation V=m/(C1−C2), wherein C1 is a concentration of the analyte at the beginning of a therapy period and C2 is a concentration of the analyte at the end of the therapy period and m is the overall mass of the analyte removed between the two measured concentrations and/or its integral.

Preferably, for the purpose of detecting a fluid-related transport impairment, also the following method steps are carried out: measuring the solution-side concentration of a desired analyte present in the solution; correlating an extinction signal and/or at least one extinction value with the measured concentration; measuring the extinction signal and/or at least one extinction value as a function of a time elapsing during a therapy and obtaining a continuously available measurement signal; calculating a concentration signal and/or at least one concentration value at predetermined discrete points in time from the continuously available measurement signal;

comparing the calculated concentration signal and/or at least one concentration value with the measured concentration; and determining if there exists a transport impairment if the signals and/or values compared to each other are dissimilar.

Preferably, the solution side is a dialysis liquid side.

Preferably, the method is carried out under the condition of a preset clearance of 100% during the therapy period.

Preferably, the solution-side concentration of the analyte as well as the blood-side concentration of the analyte is measured with a preset clearance of 100%.

Preferably, a quantitative method and/or a pH-shift method is carried out.

Preferably, an extinction signal and/or at least one extinction value is/are measured simultaneously with the quantitative method.

Preferably, an extinction signal and/or extinction value already determined during the execution of the quantitative method is/are adopted.

Preferably, the concentration value is correlated with the extinction signal and/or the at least one extinction value via a conversion factor and/or a formula relationship.

Preferably, the removed overall mass is determined by forming an integral from the calculated values.

Preferably, for the determination of the surplus water contained in a body, also the following steps are further carried out: determining the total amount of water, the total weight and the fat content of the body; determining the lean body mass by subtracting the fat content and the total amount of water from the total weight; dividing the lean body mass in a predetermined solid portion/component and a predetermined liquid portion/component, said liquid portion/component being seen as the optimum quantity of liquid; equating the determined distribution volume and the total amount of water of the body; and determining the amount of surplus water by subtracting the optimum quantity of liquid from the determined distribution volume.

Alternatively preferred, for the determination of a surplus water contained in a body, also the following steps are carried out: determining the total amount of water and of the total weight of the body; determining the lean body mass by a direct measurement using a predetermined creatinine-related kinetic model; dividing the lean body mass in a predetermined solid portion/component and a predetermined liquid portion/component, with said liquid portion/component being seen as a optimum quantity of liquid; equating the determined distribution volume and the total amount of water of the body; and determining the amount of surplus water by subtracting the optimum quantity of liquid from the determined distribution volume.

### BRIEF DESCRIPTION OF THE DRAWINGS

The invention is best understood from the following detailed description when read in connection with the accompanying drawings. Included in the drawings are the following figures:

FIG. 1 shows with a curve (a) a schematic illustration of a progression of the concentration of an analyte in a used dialysis liquid as a function of the time, with a curve (b) a schematic illustration of a progression of a signal such as for instance the absorbance of a sensor as a function of the time, and with a curve (c) an example of a calibration in which a concentration value is utilized at the beginning of a therapy for calculating concentrations of the analyte from the measurement signal;

FIG. 2 shows exemplary UV-VIS absorption spectra of a used dialysis liquid for a pH value of 7.3 and a pH value of 3.8 at four equidistant points in time of a hemodialysis therapy having a duration of 230 minutes;

FIG. 3 shows a scatter plot of extinction measurements with a pH value of 7.3 and a pH value of 3.8 while indicating the results of the regression for all points of the plot and showing a line of best fit for one point (including zero-crossing);

FIG. 4 shows a scatter plot between extinction values at 254 nm and 290 nm and with a pH value of 7.3;

FIG. 5 shows a schematically drafted illustration for an external calibration;

FIG. 6 is a schematic illustration of a typical signal progression of an optical extinction during a dialysis with a calibration point at the beginning of the therapy, with subsequently calculated points of concentrations in the measurement signal as well as the integral under the function or points or the measurement signal course;

FIG. 7 is an illustration for explaining a following or tracking method for the determination of a distribution volume;

FIG. 8 is an illustration of a signal progression of an optical extinction measurement in a dialysis liquid as well as a real development of the blood-side concentration of the analyte in the event of a transport problem at the dialyzer membrane;

FIG. 9 is a schematic illustration with respect to the determination of surplus water present in a dialysis patient as well as to the total amount of body water (fluid management); and

FIG. 10 is a conversion of the illustration in FIG. 9.

### DETAILED DESCRIPTION OF THE PREFERRED EMBODIMENTS

In the subsequent description of the Figures, identical steps, elements and/or components or steps, elements and/or components having the same effect are identically designated and/or denoted with the same reference numerals in the individual Figures and are expediently not described in redundant manner. In those cases where a subsequent exemplary embodiment functionally corresponds to at least one preceding embodiment, i.e. corresponding functions, arrangements and/or method-related procedures or operating sequences are equally comprised, only the differences will be discussed.

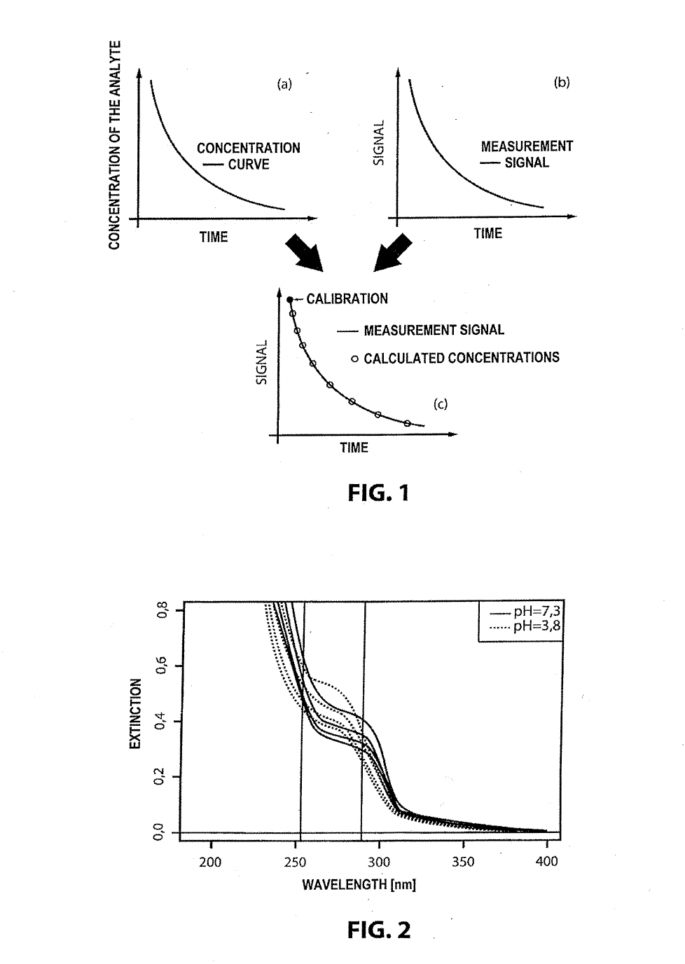

FIG. 1 shows under (a) a schematic illustration of a progression of the concentration of an analyte in a used dialysis liquid as a function of the time, under (b) a schematic illustration of a progression of a signal such as for instance the absorbance of a sensor as a function of the time and under (c) an example of a calibration in which a concentration value is utilized at the beginning of a therapy for calculating concentrations of the analyte from the measurement signal.

Thus, FIG. 1 shows in a simplified manner three different curves. Curve (a) illustrates the course of the concentration of an analyte and curve (b) illustrates the course of a measurement signal. The measurement signal and the analyte are linked in such a manner that the analyte determines a part of the measurement signal. The remaining amount of the measurement signal is determined by other, interfering substances, so that a direct conversion of the measurement signal into the correct concentration of a particular analyte is not possible in the first instance. In practice, additives are used in order to allow a unique allocation. Even if the concentration of an analyte cannot be calculated from the single available measurement signal, there are situations in which the measurement signal and the concentration of the analyte have the same or identical decay kinetics and can be linked with each other in this way. In this case, it is possible at any time to calculate further concentrations from the measurement signal which so to speak is continually available.

FIG. 1 shows with curve (c) such a case in which one calibration is sufficient for converting the measurement signal into a concentration. An initial calibration at the beginning of a therapy thus allows to avoid effortful measurements of the concentration. In doing so, a calibration point may absolutely be determined by one of the quantitative methods which are based on additives or by any other established methods, as long as following or subsequent concentration values can be calculated from the measurement signal.

FIG. 2 shows exemplary UV-VIS absorption spectra (absorption spectra of that sort of spectroscopy which uses electromagnetic waves of the ultraviolet or visible light) of a used dialysis liquid for a pH value of 7.3 as well as a pH value of 3.8 at four equidistant points in time of a hemodialysis therapy which lasts for 230 minutes.

Related to FIG. 2 and according to a first exemplary embodiment, a method for calibrating a measurement signal and tracking a quantitative variable is targeted at saving additives in the measuring of creatinine and urea on the basis of a shifting of the pH value. The shifting of the pH value of a used dialysis liquid on the basis of a change in the pH value of the solvent, for instance by adding an acidic medium, allows to perform a calculation of creatinine and urea concentrations. To this end, e.g. two extinction values are detected at a wavelength of 254nm while adding an additive between the measurement of the first and the second extinction value. The additive has such an influence on the second extinction signal that the difference between the first and second value or signal is proportional to the concentration of the analyte. The addition of the additive is necessary in this method for every concentration measurement.

According to the present exemplary embodiment of the method according to aspects of the invention, the frequency of adding the additive can be reduced.

As shown in FIG. 2 by way of example, for instance 4 pairs of UV-VIS spectra (among 16) are illustrated, which have been taken and recorded in a dialysis therapy. Four of the illustrated eight spectra relate to a pH value of 7.3, and four other of the illustrated eight spectra relate to a pH value of 3.8. As can be seen, the individual spectra correlate to a high degree in terms of their decay behavior or decay kinetics.

According to the present exemplary embodiment, the extinction value or a plurality of required extinction values is determined or calculated from the measurement for the pH value of 7.3 by selection or on the basis of the special wavelength of 254 nm, by carrying out an initial calibration of the extinction values at the pH value of 3.8 from the measurement of the extinction value at the pH value of 7.3 and by checking the extinction values at the pH value of 7.3 and the pH value of 3.8 in terms of a linear or an at least sufficiently linear connectedness, and whenever there is a linear or an at least sufficiently linear connectedness.

In other words, a first measurement signal course obtained for a first quantitative variable or a first pH value is selected from a plurality of measurement signal courses obtained for the first quantitative variable or the first pH value and for at least one second quantitative variable or a second pH value, a calibration is carried out for at least one measured value, for instance an extinction value, of the second measurement signal course with the second quantitative variable or second pH value from the measurement of the corresponding measured value with the first quantitative variable or first pH value, all measured values obtained with the first quantitative variable or first pH value and the second quantitative variable or second pH value are checked in terms of a linear or an at least sufficiently linear connectedness (i.e. a connectedness present in an approximate form), and whenever there is a linear or an at least sufficiently linear connectedness, at least one subsequent measured value for the second quantitative variable or second pH value is calculated from the first measurement signal course for the first quantitative variable or first pH value.

The calibration may be outlined here such that two predetermined and corresponding points or measured values, for instance extinction values, preferably at the beginning of a therapy and hence at the outset of respective measured curves, are correlated or made congruent with each other on respectively associated measurement signal courses, i.e. are superimposed so to speak. If these two points are congruent, a match of the individual decay kinetics of the individual measured curves can be identified. In the case of measuring extinction values, the first and the second measurement signal course is based preferably on the same wavelength as the one of the analyzing light which is used for the determination of the extinction values.

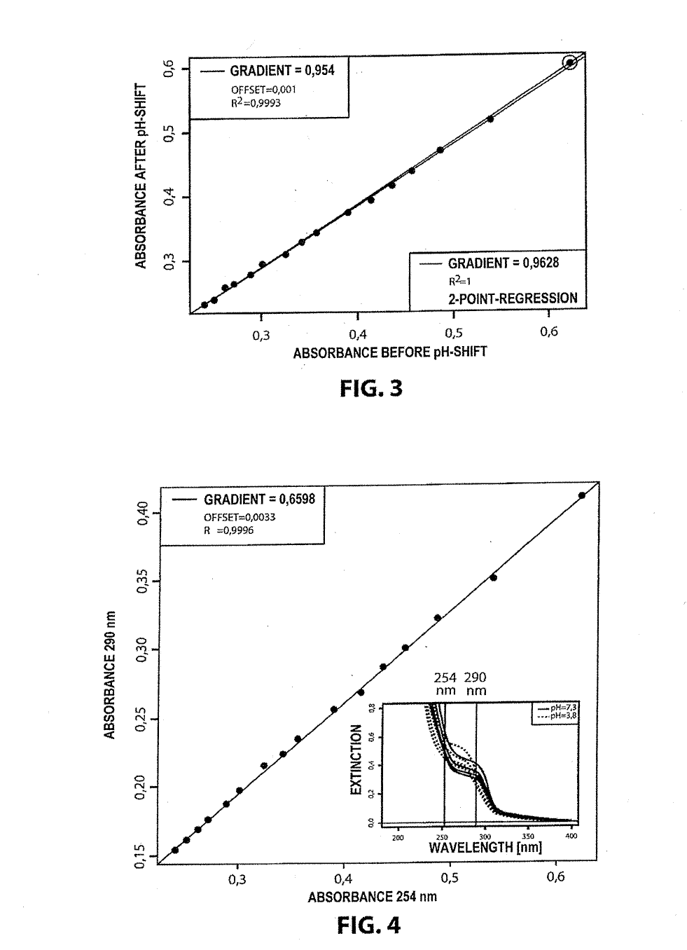

For a further explanation of the checking of linearity, FIG. 3 shows a scatter plot of extinction measurements at a pH value of 7.3 and a pH value of 3.8 while indicating the results of the regression for all points of the plot and showing a line of best fit for one point (inclusive zero-crossing).

Specifically, FIG. 3 shows a scatter plot between the extinction values at pH 7.3 and pH 3.8 at the wavelength 254 nm for different points in time in the dialysis. In addition, two regression analyses are depicted. A first line of best fit was calculated on all data points, a second one was calculated with the aid of the encircled points and the zero-crossing.

As can be seen in FIG. 3, both lines of best fit show a high degree of congruence. Whether or not the calibration is based on all points or only one point, a 2-point calibration allows to contrapose each extinction value at pH 7.3 with a corresponding pH value of 3.8. The gradient of the line of best fit is a patient-specific calibration factor in this case. From this it follows that the determination of the concentration requires a substantially smaller use of additives; furthermore, said use has to be performed only at the beginning of a therapy. The number of the measuring points which should be preferably used for the calibration depends on the respective application and may contain one or more data points depending on the implementation/design.

In a second exemplary embodiment of the method according to aspects of the invention, a further close connection or a further strong correlation between extinction values is utilized. Here, a free choice of the analyze wavelength of the measuring system is ensured on the basis of co-linearities between individual wavelengths. This makes it possible to use various light sources, giving the chance to achieve a considerable cost benefit.

FIG. 4 shows an exemplary scatter plot between extinction values at 254 nm and 290 nm and with a pH value of 7.3, in which a very strong correlation can be seen again. This allows to transfer a signal or calibration (which has been initially measured or performed with the (analyze light) wavelength of 254 nm) to the wavelength of 290 nm. This offers the opportunity to use light sources for the tracking or tracking process which have a significantly higher service life.

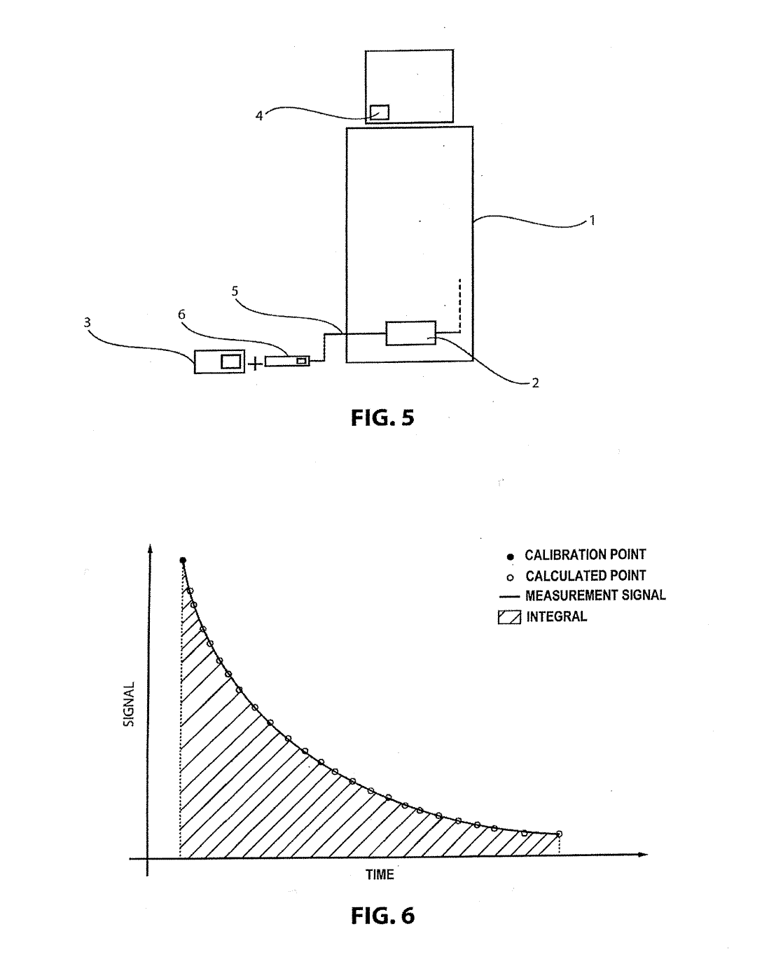

FIG. 5 shows a schematically outlined illustration for an external calibration of a trackable measuring system according to a third exemplary embodiment.

In the above, methods for determining the concentration of selected substances by a single calibration and a subsequent “tracking” of a measurement signal have been described, wherein the required calibrations are carried out by internal components of a machine. In principle, a calibration may also be performed with an external device.

According to FIG. 5, an external device 3 which is configured e.g., as an external sensor, here for instance in combination with a test strip 6, is capable of measuring a used dialysis liquid flowing out from the outlet 5 of a dialysis machine 1. The concentration of a selected analyte can be read out by the external device 3 and entered in the dialysis machine. This may be carried out either by a manual input or with an automated transmission via a wireless and/or wired interface (for instance LAN, WLAN). The matching between a relative measurement signal and a quantitatively detected variable may then be carried out within the machine in accordance with a rule deposited beforehand, for example. The dialysis machine 1 further comprises an (internal) sensor 2 and a display device 4 for displaying data and/or values. This allows the dialysis machine 1 to output further concentration values of the analyte without any additional use of other techniques and/or devices.

FIG. 6 shows a schematic illustration of an exemplary typical signal progression of an optical extinction (signal, for instance the absorbance along the ordinate versus the time along the abscissa) during a hemodialysis therapy, with a calibration point at the beginning of the therapy, with subsequently calculated points of concentrations in the measurement signal as well as the integral under the function or points or the measurement signal course, according to a fourth exemplary embodiment.

The previously described tracking method allows to calculate further concentrations from the measurement signal, which form the basis to calculate an integral which in the end results in the total amount of the selected analyte being removed during the dialysis. This removed total amount of an analyte can be used for the measurement of further variables using further technologies.

By way of example, the previously described tracking method can be used for the quantitative measurement of a blood-side concentration during a dialysis. By reducing the blood flow, for instance, it is possible to associate the concentration of a selected substance in a used dialysis liquid with a concentration of said substance in the blood, giving the chance to achieve a clearance of 100% in this case. In other words, the selected substance has the same concentration in the blood and in the used dialysis liquid and hence its concentration at the blood outlet (BA) corresponds to that at the dialysis liquid inlet (DE).

The derivation from the mass balance (simplified while omitting the ultrafiltration rate (UFR)):

Q_{b}·BE+Q_{d}·DE=Q_{b}·BA+Q_{d}·DA (equation 1)

wherein Q_{b} is the blood flow, Q_{d} is the dialysis liquid flow, BE is the blood inlet concentration, DE is the dialysis liquid inlet concentration, BA is the blood outlet concentration, and DA is the dialysis liquid outlet concentration, results in the following after resolving into the blood inlet concentration BE:

BE=BA+Q_{d}/Q_{b}19 (DA−DE) (equation 2)

In the case of a clearance of 100%, i.e. with a low blood flow, the blood outlet concentration gets close to the dialysis liquid inlet concentration:

BE=DE+Q_{d}/Q_{b}·(DA−DE) (equation 3)

If a sought substance is not present in the fresh dialysis liquid, i.e. if its concentration DE=0, the result is as follows:

BE=Q_{d}/Q_{b}·DA (equation 4)

On the basis of additional assumptions, further variables can be determined therefrom, such as the blood-side distribution volume which plays an important function or role in the dialysis when determining the dialysis dosage. In this respect, the previously described tracking method can also be used for the determination of the blood-side distribution volume.

By way of example, it is possible to calculate the distribution volume V by a combination of the detection of the blood-side concentration of a selected substance with the previously described tracking method and of the determination of the removed overall mass of the selected substance:

C₁·V−C₂·V=m equation (5)

V=m/(C1−C2) equation (6)

wherein C1 and C2 are two blood concentrations preferably at the beginning and at the end of a therapy and m is the overall mass of the substance withdrawn or removed between the two measured concentrations, i.e. its integral.

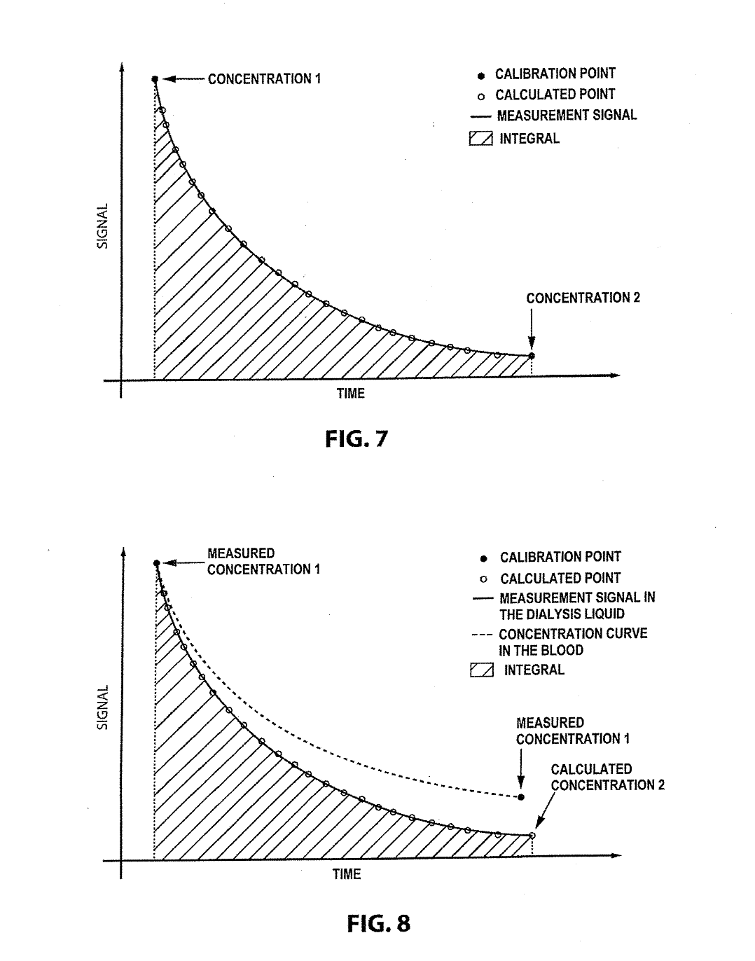

In the context mentioned above, FIG. 7 shows an illustration for explaining a tracking method for the determination of a distribution volume; specifically, it shows the essential measurands which are required for calculating the distribution volume V.

On the understanding that a clearance of 100% is adjusted between the dialysis liquid side and the blood side during a therapy treatment by suitably setting the blood flow (for instance to 50 ml/min) and the dialysis liquid flow (for instance to 500 ml/min), in accordance with FIG. 7 the concentration of a desired analyte is measured on the dialysis liquid side (value C₁) for calculating the distribution volume with a quantitative method, for instance a pH-shift method. As the substances from the blood completely go over into the dialysis liquid, their concentration in the dialysis liquid is the same as in the blood of the patient when the dialysis liquid flow and the blood flow are equal. Equation 4 applies for different flows. Simultaneously to the aforementioned quantitative method, an extinction signal or an extinction value is measured or an already determined extinction signal or an already determined extinction value is adopted for instance from the pH-shift method. Subsequently, the concentration value is linked or correlated with the extinction value via a conversion factor or a formula relationship. In the next step, the extinction signal is measured as a function of the therapy time; after that, a concentration signal is calculated at desired points in time from the signal which is then continuously available. Furthermore, the blood-side concentration of the analyte is measured under the pre-mentioned condition of a clearance of 100% as a value C₂ by the renewed application of a quantitative method. Then, the removed overall mass m is determined by forming an integral from the calculated values. Now, the distribution volume V can be determined with the determined variables and/or values using the above equation (6).

In case the calculated value for C₂ does not coincide with the measured value, the measuring of C₂ is suitable for detecting and identifying for instance any transport impairment mechanisms or transport problems, i.e. fluid-related transport impairments. To this end, the previously described tracking method can be used here as well.

FIG. 8 shows an illustration of a signal progression of an optical extinction measurement in a dialysis liquid as well as the actual progression of the blood-side concentration of the analyte in the event of a transport problem at the dialyzer membrane, and illustrates the essential processes and method steps for identifying transport problems during a therapy, for instance in a dialyzer.

On the understanding that a clearance of 100% is adjusted between the dialysis liquid side and the blood side during a therapy treatment by suitably setting the blood flow (for instance to 50 ml/min) and the dialysis liquid flow (for instance to 500 ml/min), in accordance with FIG. 8 the concentration of a desired analyte is measured on the dialysis liquid side (value C₁) for calculating the distribution volume with a quantitative method, for instance a pH-shift method. As any substances from the blood completely go over into the dialysis liquid, their concentration in the dialysis liquid is the same as in the blood of the patient when the dialysis liquid flow and the blood flow are equal. Again, equation 4 applies for different flows. Simultaneously to the aforementioned quantitative method, an extinction signal or an extinction value is measured or an already determined extinction signal or an already determined extinction value Is adopted for instance from the pH-shift method. Subsequently, the concentration value is linked or correlated with the extinction value via a conversion factor or a formula relationship. In the next step, the extinction signal is measured as a function of the therapy time; after that, a concentration signal is calculated at desired points in time from the signal which is then continuously available. Furthermore, the blood-side concentration of the analyte is measured under the pre-mentioned condition of a clearance of 100% as a value C₂ by the renewed application of a quantitative method. Then, the calculated concentration value is compared with the measured value. If the values compared to each other are equal, the absence of a transport problem is assumed. If the values compared to each other are not equal, the existence of a transport problem is assumed.

On the basis of the result of the above method, it is possible to ensure the cleaning performance during the therapy. By way of example, the Kt/V value representing the standard which would be normally used to this end would result in the indication of an especially good cleaning performance with existing transport problems, while the cleaning performance is in fact deteriorated, as it was not possible to remove a sufficiently high amount of uremic toxins due to the transport impairment mechanisms. However, as the cleaning performance is correctly determined by the aforementioned method and hence ensured, the system is capable of outputting an alarm in the event of a poor cleaning performance and subsequently the dialyzer may be changed or the therapy may be continued with a reduced performance until the corresponding dialysis dosage is achieved.

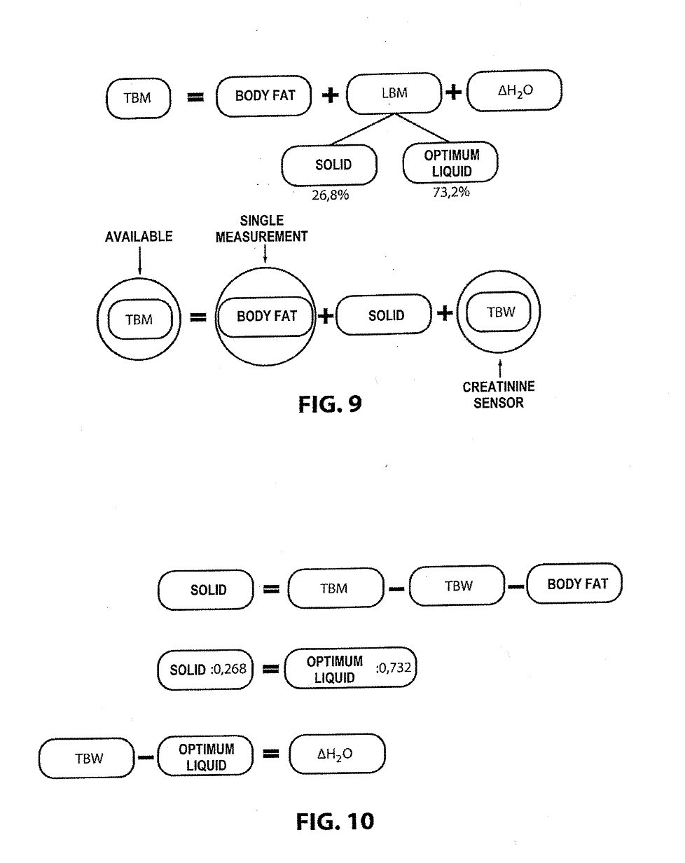

FIG. 9 shows a schematic illustration regarding the determination of the surplus water present in a dialysis patient as well as the total amount of body water (fluid management).

In this exemplary embodiment, the surplus amount of water present in a dialysis patient is identified by determining the total amount of body water (TBW; Total Body Water), the total weight of the body (TBM; Total Body Mass), the lean body mass of the body, i.e. body weight minus accumulated fat (LBM; Lean Body Mass) or the fat content or body fat of the dialysis patient. Basically, the distribution volume V of for instance creatinine may be equated to the total amount of body water TBW. The distribution volume V may be calculated with a quantitatively measuring sensor previously described. On this basis, the total weight of the body can be equated to the sum of body fat, lean body mass LBM and surplus water ΔH₂O. Using a relationship which is known per se and says that the lean body mass LBM for a human can be divided into a fixed ratio of 26.8% solid components and 73.2% optimum liquid, the conversion of the illustration in FIG. 9 shown in FIG. 10 can be derived with respect to the fluid management.

In other words, the amount of the water which has to undergo an ultrafiltration can be identified with a measurement of the total weight TBM, the fat content and the total amount of body water TBW of the body. On this basis, the optimum amount of water can be calculated and with the knowledge of the total amount of body water TBW the amount of surplus water can be determined.

Alternatively, the determination of the surplus amount of water is possible by a direct measurement of the lean body mass LBM. When using a creatinine sensor as a quantitative sensor, also a direct measurement of the lean body mass LBM by use of a so-called creatinine kinetic model is possible apart from the determination of the total amount of body water TBW, for example according to the paper “Lean Body Mass Estimation by Creatinine Kinetics”, Prakash R. Keshaviah et al. Journal of the American Society of Nephrology, volume 4, No. 7, 1994. Here, the measurement of the body fat content may be omitted and the optimum amount of water is obtained directly via the lean body mass LBM. The surplus amount of water can then be identified by subtracting the optimum amount of water from the total amount of body water TBW.

Thus, a method for calibrating a measurement signal and/or for tracking a quantitative variable has been described above. The method comprises the measuring of an analyte which exists in solution with a certain concentration and has predetermined decay kinetics and the generating of a quasi-continuous measurement signal having decay kinetics which at least correspond to those of the analyte. The decay kinetics of the measurement signal and the decay kinetics of the analyte are correlated using at least one predetermined calibration point of both decay curves. Subsequent concentration values of the analyte are then calculated from the measurement signal.

It goes without saying that the invention is not limited to the described exemplary embodiments and the numerical values and orders of magnitudes which have been stated in the context thereof; rather, a person skilled in the art may infer modifications and equivalents within the scope of protection defined by the following claims.

1-17. (canceled)

18. Method for determining concentration values of an analyte, comprising:
measuring an analyte which exists in a solution with a certain concentration and has predetermined decay kinetics and generating a continuous measurement signal having decay kinetics at least corresponding to those of the analyte;
correlating the decay kinetics of the measurement signal with the decay kinetics of the analyte using at least one predetermined calibration point between the decay kinetics of the measurement signal and the decay kinetics of the analyte; and
calculating subsequent concentration values of the analyte from the correlated measurement signal.

19. Method according to claim 18, further comprising:
selecting a first measurement signal course obtained for a first quantitative variable among a plurality of measurement signal courses obtained for the first quantitative variable and for at least one second quantitative variable;
carrying out a calibration for at least one measured value of a second measurement signal course with the second quantitative variable from the measurement of the corresponding measured value with the first quantitative variable;
checking all measured values obtained with the first quantitative variable and the second quantitative variable to determine linear connectedness; and
whenever linear connectedness is determined, calculating at least one subsequent measured value for the second quantitative variable from the first measurement signal course for the first quantitative variable.

20. Method according to claim 19, wherein the first quantitative variable is a first pH value, the second quantitative variable is a second pH value, and the measured values are extinction values.

21. Method according to claim 19, wherein the first and the second measurement signal courses are based on the same wavelength of an analyzing light used for the determination of the extinction values.

22. Method according to claim 18, further comprising calculating a distribution volume by:
measuring a solution-side concentration of desired analyte present in a solution;
correlating an extinction signal and/or at least one extinction value with the measured concentration;
measuring the extinction signal and/or at least one extinction value as a function of a time elapsing during a therapy and obtaining a continuously available measurement signal;
calculating a concentration signal and/or at least one concentration value at predetermined discrete points in time from the continuously available measurement signal;
measuring a blood-side concentration of the analyte;
determining a removed overall mass; and
determining the distribution volume from the determined overall mass, the solution-side concentration of the analyte and the blood-side concentration of the analyte.

23. Method according to claim 22, wherein the distribution volume is calculated in accordance with the relation
V=m/(C1−C2),
wherein C1 is a concentration of the analyte at the beginning of a therapy period, C2 is a concentration of the analyte at the end of the therapy period, and m is the overall mass of the analyte removed between the two measured concentrations and/or its integral.

24. Method according to claim 18, further comprising detecting a fluid-related transport impairment by:
measuring a solution-side concentration of a desired analyte present in a solution;
correlating an extinction signal and/or at least one extinction value with the measured concentration;
measuring the extinction signal and/or at least one extinction value as a function of a time elapsing during a therapy and obtaining a continuously available measurement signal;
calculating a concentration signal and/or at least one concentration value at predetermined discrete points in time from the continuously available measurement signal;
comparing the calculated concentration signal and/or at least one concentration value with the measured concentration; and
determining if there exists a transport impairment if the signals and/or values compared to each other are dissimilar.

25. Method according to claim 22, wherein the solution side is a dialysis liquid side.

26. Method according to claim 22, wherein a condition of a preset clearance of 100% is used during the therapy period.

27. Method according to claim 22, wherein the solution-side concentration of the analyte and the blood-side concentration of the analyte is measured with a preset clearance of 100%.

28. Method according to claim 22, wherein a quantitative method and/or a pH shift method is/are carried out.

29. Method according to claim 28, wherein the extinction signal and/or at least one extinction value is/are measured simultaneously with the quantitative method.

30. Method according to claim 28, wherein the extinction signal and/or extinction value already determined during the execution of the quantitative method is/are adopted.

31. Method according to claim 22, wherein the concentration value is correlated with the extinction signal and/or the at least one extinction value via a conversion factor and/or a formula relationship.

32. Method according to claim 22, wherein the removed overall mass is determined by forming an integral from the calculated values.

33. Method according to claim 22, further comprising determining surplus water contained in a body by:
determining a total amount of water, a total weight and a fat content of the body;
determining a lean body mass by subtracting the fat content and the total amount of water from the total weight;
dividing the lean body mass in a predetermined solid component and a predetermined liquid component, said liquid component being seen as the optimum quantity of liquid;
equating the determined distribution volume and the total amount of water of the body; and
determining the amount of surplus water by subtracting the optimum quantity of liquid from the determined distribution volume.

34. Method according to claim 22, further comprising determining surplus water contained in a body by:
determining a total amount of water and a total weight of the body;
determining a lean body mass by a direct measurement using a predetermined creatinine-related kinetic model;
dividing the lean body mass in a predetermined solid component and a predetermined liquid component, with said liquid component being seen as the optimum quantity of liquid;
equating the determined distribution volume and the total amount of water of the body; and
determining the amount of surplus water by subtracting the optimum quantity of liquid from the determined distribution volume.
